(19) United States
(12) Patent Application Publication
(10) Pub. No.: US 2010/0172852 A1
(43) Pub. Date: Jul. 8, 2010

(54) COSMETIC COMPOSITION COMPRISING AN EXTRACT OF CHINESE INSECT WAX

(75) Inventors: Sophie Favre, Paris (FR); Sophie Foucault, Colombes (FR)

Correspondence Address: YOUNG & THOMPSON, 209 Madison Street, Suite 500, Alexandria, VA, 22314, US

(73) Assignee: CHANEL PARFUMS BEAUTE, Neuilly Sur Seine (FR)

(21) Appl. No.: 12/602,473

(22) PCT Filed: May 26, 2008
(86) PCT No.: PCT/EP08/56427

§ 371 Date: Nov. 30, 2009

(30) Foreign Application Priority Data
May 31, 2007 (FR) 07 03875

(51) Int. Cl. A61K 8/92; A61Q 1/04; A61Q 1/10; A61Q 1/06

(52) U.S. Cl. 424/63; 424/64; 424/69; 424/70.7; 424/538

(57) ABSTRACT

A cosmetic composition in solid form includes 25-90% of at least one silicone oil and an extract of Ericerus pela wax. A cosmetic process for caring for and/or making up keratinous matter, includes the application to the keratinous matter of the cosmetic composition. In addition, a process of using an extract of Ericerus pela wax for structuring at least one silicone oil is described.

The present invention relates to a cosmetic composition in solid form comprising at least one silicone oil and an extract of Ericerus pela wax. The invention also relates to the use of an extract of Ericerus pela wax for structuring at least one silicone oil.

Ericerus pela is a Chinese insect belonging to the Coccoidea family. This insect is of great importance in China on account of its ability to produce wax and its high nutritional value.

Silicones or polysiloxanes are organic polymers formed from a silicon-oxygen chain ( . . . —Si—O—Si—O—Si—O— . . . ) onto which are attached groups, especially alkyls.

Silicones may especially be in the form of oils, gels, gums, emulsions or resin or elastomer powders.

It is very common practice in cosmetics to use silicone oils.

For example, volatile silicone oils afford glidance on application, and also matting of the skin and a non-greasy finish after evaporation. Low-viscosity oils give glidance without a rich greasy sensation, whereas high-viscosity oils give softness and very long-lasting glidance without a sensation of heaviness. As regards functionalized oils, especially phenylated oils, they afford gloss on account of their high refractive index.

However, it is very difficult to structure silicone oils so as to obtain solid products, since silicones are sparingly compatible with the waxes traditionally used. Now, it is advantageous to use a high proportion of silicone oils in lipsticks or solid foundations, for example, since these oils afford a range of specific feels while at the same time giving the cosmetic composition particular properties.

It is already known practice to use an extract of Ericerus pela wax in the pharmaceutical field and more particularly as an agent for combating hemorrhages, as an analgesic, as an antitussive agent, as a cicatrizing agent and as an agent for treating diarrhea (Li, World Animal Review 55: 26-33, 1985).

Patent JP 3798735 moreover describes a solid cosmetic composition comprising a solid oil including from 0.1% to 30% of an extract of Ericerus pela wax, from 5% to 97.4% of an oil other than the solid oil and from 0.1% to 70% of a powder, the percentages being expressed by weight relative to the total composition.

It is also known practice to use silicone-grafted polyamides or polyethylene waxes in silicone-rich solid compositions.

There is still need to propose novel types of solid cosmetic compositions that are rich in silicone oils, which show good stability of the solid composition over time, and which also have a sparingly tacky texture.

The Applicant has now discovered that it is possible to use an extract of Ericerus pela wax to structure silicone oils in a solid cosmetic composition comprising a high content of silicone oils.

The invention is more particularly directed toward a cosmetic composition in solid form comprising from 25% to 90% by weight of at least one silicone oil and an extract of Ericerus pela wax.

The wax derived from Ericerus pela is secreted by a waxy gland of the female insects and of the male insects. It is also known under the INCI (International Nomenclature of Cosmetic Ingredients) name Ericerus Pela Wax.

This wax, also known as Chinese insect wax, is a white or yellowish-white crystalline wax resembling spermaceti or whale wax, but is harder and also crumbly, and has a higher melting point.

To obtain Ericerus pela wax, Ericerus pela insects and their secretions can be collected and boiled in water, and the whole then extracted using a solvent such as ethyl acetate.

In addition, this wax is especially sold under the brand name Snow White Wax® by Cera Rica Noda, and also under the brand name Refined Chinese Insect Wax® by Baerlocher.

As indicated in US patent application 2005/0239904 A1, the composition of Ericerus pela wax, analyzed by gas chromatography/mass spectroscopy, comprises, for example, about 55% hexacosyl hexacosanoate, about 22% hexacosyl tetracosanoate and about 16.65% hexacosyl octacosanol.

A subject of the present invention is also a cosmetic process for caring for and/or making up keratinous matter, wherein it comprises the application to the keratinous matter of the cosmetic composition according to the invention.

The term “keratinous matter” means the skin, the lips and the integuments, including keratin fibers such as the hair, the eyelashes and the eyebrows.

A subject of the invention is also the use of an extract of Ericerus pela wax for structuring at least one silicone oil.

The term “silicone oil” means an oil comprising at least one silicon atom, and especially at least one Si—O group.

The silicone oils may be in the form of volatile oils or nonvolatile oils.

For the purposes of the invention, the term “volatile oil” means an oil capable of evaporating on contact with keratinous matter in less than one hour, at room temperature and atmospheric pressure. The volatile oils of the invention are volatile cosmetic oils, which are liquid at room temperature, and which have a nonzero vapor pressure at room temperature and atmospheric pressure, for example ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Examples of volatile silicone oils, or volatile silicones, are especially linear or cyclic silicone oils with a viscosity of less than 8 centistokes (8×10⁻⁶ mVs) and in particular containing from 2 to 10 silicon atoms and more particularly from 2 to 7 silicon atoms, these silicones optionally comprising alkyl or alkoxy groups containing from 1 to 10 carbon atoms. As volatile silicone oils that may be used in the invention, mention may be made especially of certain dimethicones with a viscosity of 5 and 6 cSt, octamethylcyclotetrasiloxane, decamethylcyclopentasiloxane, dodecamethylcyclohexasiloxane, hexamethyldisiloxane, octamethyltrisiloxane, decamethyltetrasiloxane, dodecamethylpentasiloxane, the compounds identified by the INCI names methyl trimethicone and caprylyl methicone, and mixtures thereof.

Mention may also be made of the linear volatile alkyltrisiloxanes of general formula (I)

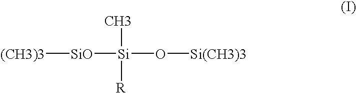

in which R represents an alkyl group containing from 2 to 6 carbon atoms, one or more hydrogen atoms of which may be replaced with a fluorine or chlorine atom.

Among the oils of general formula (I), mention may be made of: hexyl-1,1,1,3,5,5,5-heptamethyltrisiloxane, 3-butyl-1,1,1,3,5,5,5-heptamethyltrisiloxane, 3-dipropyl-1,1,1,3,5,5,5-heptamethyltrisiloxane and 3-ethyl-1,1,1,3,5,5,5-heptamethyltrisiloxane, corresponding to the oils of formula (I) for which R is, respectively, a butyl group, a propyl group or an ethyl group.

The term “nonvolatile oil” means an oil that remains on the keratinous matter for at least one hour at room temperature and atmospheric pressure, and that especially has a vapor pressure of less than 10⁻³ mmHg (0.13 Pa).

The nonvolatile silicone oils that may be used in the composition according to the invention may be nonvolatile polydimethylsiloxanes (PDMS), polydimethylsiloxanes comprising alkyl and/or alkoxy groups, which are pendent and/or at the end of a silicone chain, these groups each containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisilexanes, alkyl and/or alkoxy dimethicones, for instance bis(hydroxyethoxy)propyl dimethicone, and 2-phenylethyl trimethylsiloxysilicates.

Among these nonvolatile oils, mention may be made in particular of glossy oils.

The term “glossy oil” means an oil that has a refractive index of greater than 1.45 and preferably greater than 1.47.

Examples of glossy oils are especially phenyl silicone oils, such as those identified by the INCI name “phenyl trimethicone”, two examples of which are the silicone available under the trade name Mirasil® PTM from the company Rhodia and the silicone sold under the brand name Abil® AV 20 by Degussa, alkyl siloxysilicates such as those identified by the INCI name “phenylpropyldimethyl siloxysilicate”, an example of which is the silicone available under the trade name Silshine® 151 from the company General Electric and those identified by the INCI name “trimethyl pentaphenyl trisiloxane”, an example of which is the silicone available under the trade name DC® PH 1555 HRI from the company Dow Corning.

Glossy oils that may also be mentioned include the fluoro silicones identified by the INCI name perfluorononyl dimethicone, an example of which is the silicone available under the trade name Pecosil® FS (FSU, FST, etc.) from the company Phoenix, and another example is the silicone available under the trade name Biosil® Basics (Fluorosil LF, 14, etc.) from the company Biosil Technologies.

The expression “structuring silicone oils” means gelling and/or rigidifying and/or solidifying these oils, in order to impart good stability and absence of exudation or softening over time, at room temperature, to cosmetic products comprising them.

It is considered that the stability of the product is satisfactory when the penetration index as defined under the conditions of Example B is less than 100×10⁻¹ mm, preferably less than 70×10⁻¹ mm and more preferentially less than 50×10⁻¹ mm; and/or when the breaking index as defined under the conditions of Example B is greater than 100 g, preferably greater than 200 g and more preferentially greater than 500 g. The breaking index is the weight required to crush the cast.

In one preferred embodiment, the silicone oils are glossy oils such as phenyl silicones or alkyl siloxysilicates, and mixtures thereof.

In another preferred embodiment, the silicone oil is chosen from phenyl trimethicone, phenylpropyldimethyl siloxysilicate, bis(hydroxyethoxy)propyl dimethicone, and mixtures thereof.

The silicone oils according to the invention represent from 25% to 90%, preferably from 30% to 80% and even more preferably from 40% to 75% by weight relative to the weight of the total composition.

In addition, in one preferred embodiment, the extract of Ericerus pela wax represents from 5% to 35% and preferably from 10% to 20% by weight relative to the weight of the total composition.

The composition according to the invention is preferably a facial make-up composition and more preferably a make-up composition for lips. It may be in the form of a pencil, a mascara, an eyeliner, a foundation, a lip gloss, a lipstick, a lipcare stick, a body makeup product, an eyeshadow, a makeup rouge, a concealer product, a facial care product or a body care product. Lipsticks are preferred according to this invention.

According to one preferred embodiment, the composition is in cast form, especially in a jar or dish.

As a variant, said composition is in the form of a stick.

Another subject of the invention is the cosmetic use of said composition for caring for and/or making up keratinous matter.

The composition according to the invention is advantageously in anhydrous form, in the sense that it contains less than 5% by weight of water or even less than 1% by weight of water, or even no water at all. As a variant, it may be in the form of an oil-in-water, water-in-oil or multiple (W/O/W, O/W/O, etc.) emulsion or in the form of a hydrodispersion (dispersion of oil in a polar phase, especially an aqueous phase, in the absence of emulsifier).

In addition to the silicone oil as described previously, the composition according to the invention may contain at least one other oil.

Other oils that may especially be mentioned include: linear or branched hydrocarbons of mineral or synthetic origin, synthetic (poly)esters and (poly)ethers and in particular (poly)esters of C₆-C₂₀ acids and of C₆-C₂₀ alcohols that are advantageously branched, such as isononyl isononanoate, plant oils, branched and/or unsaturated fatty acids, branched and/or unsaturated fatty alcohols, fluoro oils, and also mixtures thereof.

The term “hydrocarbon-based oil” means an oil containing only hydrogen and carbon atoms. Volatile hydrocarbon-based oils may be chosen from hydrocarbon-based oils containing from 8 to 16 carbon atoms, and especially branched C₅-C₁₆ alkanes (also known as isoparaffins), for instance isododecane (also known as 2,2,4,4,6-pentamethylheptane), isodecane or isohexadecane.

The term “fluoro oils” means an oil containing at least one fluorine atom and not containing any silicon atoms, such as nonafluoromethoxybutane or perfluoromethyl-cyclopentane, perfluorodimethylcyclohexane, perfluoro-perhydrophenanthrene, perfluorodecalin, and mixtures thereof, without this list being limiting.

In one preferred embodiment, the other oil is an ester chosen from isononyl isononanoate, diisostearyl malate, octyldecvl neopentanoate, 2-ethylhexyl palmitate, and mixtures thereof; and more preferably, the ester is isononyl isononanoate.

The fatty phase of this composition may also contain at least one wax other than the Chinese insect wax described previously. The term “wax” means a fatty substance with a melting point of greater than 30° C. and generally less than 100° C., which is liquid under the conditions of preparation of the composition (advantageously between 50 and 95° C.) and which has in the solid state an anisotropic crystal organization. Examples of waxes are especially plant, animal, mineral or synthetic waxes, the latter advantageously possibly being hydrocarbon-based waxes or silicone waxes. Mention may thus be made of carnauba wax, candelilla wax, rice wax, beeswax (Cera alba) and polyethylene wax, which are optionally functionalized, and paraffin wax, and also ozokerite, microcrystalline waxes, linear C₁₄-C₂₂ fatty alcohols and triesters of C₈-C₂₀ acids and of glycerol, such as glyceryl tribehenate, solid pentaerythritol esters such as pentaerythrityl tetrastearate and pentaerythrityl tetrabehenate, and mixtures thereof, without this list being limiting. Mention may also be made of the acetylated glycol stearate sold by the company Vevy under the trade name Cetacene®. Mention may be made of silicone waxes such as those sold by the company Wacker under the trade name Reisil® CD 7026 and Belsil® CDM 3526.

The fatty phase of this composition may also contain a pasty fatty substance or a butter characterized by a melting point of between 25 and 45° C. Mention may be made especially of the ester sold by the company Croda under the trade name Super Sterol Ester®, pentaerythrityl tetracocoate, the hydrogenated coconut oil sold by the company Prod'Hyg under the trade name Hydrobase® 32/34, and plant butters.

The fatty phase of this composition may also advantageously contain at least one lipophilic gelling agent.

Examples of lipophilic gelling agents are especially silicone polymers and more particularly organopolysiloxane elastomers. Among these, mention may be made of at least partially crosslinked polymers resulting from the reaction of an organopolysiloxane bearing unsaturated groups, such as vinyl or allyl groups, located at the end or in the middle of the chain, preferably on a silicon atom, with another reactive silicone compound such as an organohydrogenopolysiloxane. These polymers are usually available in the form of a gel in a volatile or nonvolatile silicone solvent or in a hydrocarbon-based solvent. Examples of such elastomers are especially sold by the company Shin-Etsu under the trade names KSG-6®, KSG-16®, KSG-31, ®KSG-32®, KSG-41®, KSC-42®, KSG-43® and KSG-44®, and by the company Dow Corning under the trade names DC® 9040 and DC® 9041. Other examples of elastomers are sold by the company Wacker under the name Belsil® RG100. Another oily gelling agent is a silicone polymer, obtained by self-polymerization of an organopolysiloxane functionalized with epoxy and hydrosilyl groups, in the presence of a catalyst, which is commercially available from the company General Electric under the trade name Velvesil®125. Another lipophilic gelling agent is a cyclic dimethicone/vinyl dimethicone copolymer such as the product sold by the company Jeen under the trade name Jeesilc® PS (including PS-VH, PS-VHLV, PS-CM, PS-CMLV and PS-DM). Another type of lipophilic gelling agent consists of copolymers of styrene and of olefins such as ethylene, propylene and/or butylene, optionally combined with silicone or hydrocarbon-based solvents, as described in particular in patent application WO 98/38981 and in U.S. Pat. No. 6,309,629. They especially comprise the block terpolymer-based gelling agents available from the company Penreco under the trade name Versagel. As a variant, the lipophilic gelling agent may be a bentone, or a silica with a very large specific surface area, namely from 300 to 1000 m²/g, obtained by pyrogenation and then substitution of the OH groups with CH₃ (for example available under the trade name Aerosil®R872 or Aerosil® R972 from the company Degussa), or obtained via a sol-gel route (available under the trade name Aerogel® from the company Cabot). Inulin derivatives may also be used, such as the product available under the trade name Rheopearl ISK from the company Miyoshi.

The composition used according to the invention may also contain at least one film-forming polymer, in particular a polymer capable of affording staying power and/or transfer-resistance properties to the makeup result imparted by the composition.

This film-forming polymer may be lipophilic. It may especially be an optionally urethane- or fluoro- or acrylate-modified silicone polymer, such as the (meth)acrylate silicones sold by Shin-Etsu under the trade names KP-545®, KP-561° and KP-562® or the polymers sold by the company Dow Corning under the trade names DC® FA 4002 ID and DC® FA 4001 CM. Other examples of film-forming polymers are silicone resins and in particular MQ resins such as trimethyl siloxysilicates and MT resins such as silsesquioxane derivatives and especially polymethylsilsesquioxanes, sold especially by the company Shin-Etsu, and also polypropylsilsesquioxane sold by the company Dow Corning under the trade name DC® 670, phenylpropyl polysilsesquioxane sold by the company Wacker under the trade name Belsil® SPR45VP or a copolymer of C₃₀ alkyl chain/resin of the silsesquioxane type. Another example consists of fluoro silicone polymers identified by the INCI name trifluoropropyldimethylsiloxy triethylsiloxysilicate such as those sold by the company General Electric under the trade name X566-B8226® and XS66-B8236®. It is also possible to use as film-forming polymers bioadhesive polymers obtained, for example, by polycondensation of dimethiconol and of MQ silicate resin in a solvent such as heptane, which are especially sold by the company Dow Corning under the trade names DC®7-4405 low tack and DC® 7-4505 high tack. Other examples of film-forming polymers are cyclic polyolefins such as polycyclopentadiene, especially sold by the company Kobo under the trade name Koboguard 5400, or alternatively polydicyclopentadiene. Even further examples of film-forming polymers are copolymers of vinylpyrrolidone (VP) and/or of linear olefins such as VP/hexadecene and VP/eicosene copolymers, including Antaron® V216 and Antaron® V220 from the company ISP, or alternatively ethylene/vinyl acetate copolymers such as AC 400 from the company Baerlocher. Other film-forming polymers that may be used in this invention are polyacrylates such as poly(ethyl acrylate) sold especially by the company Creations Couleurs under the trade name Creasil® 7 ID.

As a variant or in addition, when it is in the form of an emulsion or a hydrodispersion, the composition according to the invention may contain at least one hydrophilic film-forming polymer and/or one or more hydrophilic gelling agents.

This composition may also contain one or more surfactants preferably chosen from nonionic surfactants, such as optionally polyethoxylated sorbitan esters, fatty acid esters of glycerol, fatty acid esters of sucrose, fatty acid esters of polyethylene glycol, polyether-modified polysiloxanes, fatty alkyl ethers of polyethylene glycol, alkylpolyglycosides and hydrogenated lecithin, without this list being limiting. It is preferred for the composition according to the invention not to contain any anionic, cationic or amphoteric surfactant. The term “surfactant” means a compound identified as such in the McCutcheon dictionary.

In a preferred embodiment, however, the composition of this invention is free from surfactant.

The composition used according to the invention may also contain at least one filler. This term means any mineral or organic, natural or synthetic particle of any shape (especially spherical or lamellar), which is insoluble in the composition. Examples of fillers are talc, mica, silica, kaolin, boron nitride, starch, starch modified with octenylsuccinic anhydride, polyamides, silicone resins, silicone elastomer powders and acrylic polymer powders, in particular of poly(methyl methacrylate). The fillers may especially be constituted of several layers of different chemical nature and/or physical form, and may especially be in the form of leaflets coated with spherical fillers. They may be modified by means of various surface treatments with agents such as silicones, isopropyl titanium triisostearate, perfluoroalkyl phosphate, triethoxycaprylylsilane, etc., or mixtures thereof.

The composition may also contain at least one dyestuff chosen from water-soluble or liposoluble dyes, fillers having the effect of coloring and/or opacifying the composition and/or the skin, such as pigments, nacres and lakes (water-soluble dyes adsorbed onto an inert mineral support), and mixtures thereof. These dyestuffs may optionally be surface-treated with a hydrophobic agent such as silanes, silicones, fatty acid soaps, C₉-C₁₅ fluoroalcohol phosphates, acrylate/dimethicone copolymers, C₉-C₁₅ fluoroalcohol phosphate/silicone mixed copolymers, lecithins, carnauba wax, polyethylene, chitosan and optionally acylated amino acids such as lauroyllysine, disodium stearoyl glutamate and aluminum acyl glutamate. The pigments may be mineral or organic, and natural or synthetic. Examples of pigments are especially iron oxide, titanium oxide, chromium oxide or zinc oxide, ultramarine blue, Prussian blue, carbon black, pigments of D&C type such as D&C Red 33 Aluminum Lake®, and also composite pigments and goniochromatic pigments, pearlescent pigments, interference pigments, photochromic pigments or thermochromic pigments, without this list being limiting. The nacres may be chosen from those conventionally present in makeup products, such as mica-titanium dioxide.

The composition according to the invention may also contain active agents and in particular antioxidants such as ascorbic acid alkyl or phosphoryl esters, or alternatively tocopherol and esters thereof; sequestrants such as EDTA salts; pH regulators; preserving agents; and fragrances. According to a preferred embodiment of this invention, the composition does not comprise more than 1% by weight of any alcohol such as ethanol. Most preferably, it is free of any alcohol such as ethanol.

This composition may also contain at least one UV-screening agent chosen from organic and mineral screening agents, and mixtures thereof. Organic screening agents that may especially be mentioned include dibenzoylmethane derivatives (including butylmethoxydibenzoylmethane), cinnamic acid derivatives (including ethylhexyl methoxycinnamate), salicylates, para-aminobenzoic acids, β,β′-diphenylacrylates, benzophenones, benzylidenecamphor derivatives, phenylbenzimidazoles, triazines, phenyl-benzotriazoles and anthranilic derivatives. Mineral screening agents that may especially be mentioned include screening agents based on mineral oxides in the form of coated or uncoated pigments or nanopigments, and in particular based on titanium dioxide or zinc oxide.

Examples of such adjuvants are mentioned especially in the CTFA dictionary (International Cosmetic Ingredient Dictionary and Handbook published by The Cosmetic, Toiletry and Fragrance Association, 11th edition, 2006).

The invention will now be illustrated by the nonlimiting examples that follow.

### EXAMPLES

### A. Cosmetic Compositions

The compositions below, in which the ingredients are identified by their INCI names and the amounts thereof are indicated as weight percentages, were prepared in a manner conventional for those skilled in the art.

### Example 1

### Lipstick

| | |
|---|---|
| Ericerus Pela Wax (Snow White Wax ®) | 11.70% |
| Phenyl trimethicone (Abil ® AV 20) | 24.20% |
| Bis(hydroxyethoxy)propyl dimethicone | 24.20% |
| (DC ® 2-5562 carbinol fluid) | |
| Isononyl isononanoate (Wickenol ® 151) | 22.40% |
| Titanium dioxide (titanium oxide W 877 ®) | 2.80% |
| D&C Red 33 Aluminum Lake (6533 D&C red AL lake ®) | 2.20% |
| Mica & iron oxides (cloisonne cerise flambé ®) | 12.50% |

### Example 2

### Lipstick

| | |
|---|---|
| Ericerus Pela Wax (Refined Chinese Insect Wax ®) | 12.50% |
| Phenyl trimethicone (Abil ® AV 20) | 24.10% |
| Bis(hydroxyethoxy)propyl dimethicone | 24.10% |
| (DC 2-5562 carbinol fluid ®) | |
| Isononyl isononanoate (Wickenol ® 151) | 21.80% |
| Titanium dioxide (titanium oxide W 877 ®) | 2.80% |
| D&C Red 33 Aluminum Lake (6533 D&C red AL lake ®) | 2.20% |
| Mica & iron oxides (cloisonne cerise flambé ®) | 12.50% |

### Example 3

### Lipstick

| | |
|---|---|
| Ericerus Pela Wax (Snow White Wax ®) | 11.70% |
| Phenyl trimethicone (Abil ® AV 20) | 35.40% |
| Bis(hydroxyethoxy)propyl dimethicone | 35.40% |
| (DC 2-5562 carbinol fluid ®) | |
| Titanium dioxide (titanium oxide W 877 ®) | 2.80% |
| D&C Red 33 Aluminum Lake (6533 D&C red AL lake ®) | 2.20% |
| Mica & iron oxides (cloisonne cerise flambé ®) | 12.50% |

### Example 4

### Lipstick

| | |
|---|---|
| Ericerus Pela Wax (Snow White Wax ®) | 11.70% |
| Phenyl trimethicone (Abil ® AV 20) | 70.80% |
| Titanium dioxide (titanium oxide W 877 ®) | 2.80% |
| D&C Red 33 Aluminum Lake (6533 D&C red AL lake ®) | 2.20% |
| Mica & iron oxides (cloisonne cerise flambé ®) | 12.50% |

### Example 5

### Lipstick

| | |
|---|---|
| Ericerus Pela Wax (Snow White Wax ®) | 10.00% |
| Synthetic paraffin (Sasol ® Wax C80) | 3.00% |
| Phenyl trimethicone (Abil ® AV 20) | 24.00% |
| Bis(hydroxyethoxy)propyl dimethicone | 24.00% |
| (DC 2-5562 carbinol fluid ®) | |
| Isononyl isononanoate (Wickenol ® 151) | 21.50% |
| Titanium dioxide (titanium oxide W 877 ®) | 2.80% |
| D&C Red 33 Aluminum Lake (6533 D&C red AL lake ®) | 2.20% |
| Mica & iron oxides (cloisonne cerise flambé ®) | 12.50% |

### Example 6

### Lipstick

| | |
|---|---|
| Ericerus Pela Wax (Snow White Wax ®) | 11.70% |
| Phenylpropyldimethyl siloxysilicate | 24.20% |
| (Silshine ® 151) | |
| Bis(hydroxyethoxy)propyl dimethicone | 24.20% |
| (DC 2-5562 carbinol fluid ®) | |
| Isononyl isononanoate (Wickenol ® 151) | 22.40% |
| Titanium dioxide (titanium oxide W 877 ®) | 2.80% |
| D&C Red 33 Aluminum Lake (6533 D&C red AL lake ®) | 2.20% |
| Mica & iron oxides (cloisonne cerise flambé ®) | 12.50% |

### Example 7

### Lipstick

| | |
|---|---|
| Ericerus Pela Wax (Refined Chinese Insect Wax ®) | 13.40% |
| Phenyl trimethicone (Mirasil PTM ®) | 47.20% |
| PVP/eicosene Copolymer (Antaron ® V220) | 4.00% |
| Diisostearyl malate (Salacos ® 222) | 17.90% |
| D&C Red 7 Calcium Lake (Rubine Lake C 19003 ®) | 5.18% |
| Titanium dioxide (titanium oxide W 877 ®) | 4.45% |
| D&C Red 33 Aluminum Lake (6533 D&C red AL lake ®) | 0.55% |
| D&C Red 7 Calcium Lake (Rubine Lake C 19025 ®) | 0.81% |
| FD&C Yellow No. 6 Aluminum Lake | 0.96% |
| (FD&C Yellow 6 Lake (C70.52.70)) | |
| Mica & Aluminum hydroxide (Excel Mica JP-2 ®) | 5.55% |

### Example 8

### Lipstick

| | |
|---|---|
| Ericerus Pela Wax (Refined Chinese Insect Wax ®) | 13.40% |
| Phenyl trimethicone (Mirasil PTM ®) | 47.20% |
| PVP/eicosene Copolymer (Antaron ® V220) | 4.00% |
| Diisostearyl malate (Salacos ® 222) | 17.90% |
| D&C Red 7 Calcium Lake (Rubine Lake C 19003 ®) | 5.18% |
| Titanium dioxide (titanium oxide W 877 ®) | 4.45% |
| D&C Red 33 Aluminum Lake (6533 D&C red AL lake ®) | 0.55% |
| D&C Red 7 Calcium Lake (Rubine Lake C 19025 ®) | 0.81% |
| FD&C Yellow No. 6 Aluminum Lake | 0.96% |
| (FD&C Yellow 6 Lake (C70.52.70)) | |
| Mica & Titanium dioxide (Flamenco sparkle orange ®) | 5.55% |

### Example 9

### Lipstick

| | |
|---|---|
| Ericerus Pela Wax (Refined Chinese Insect Wax ®) | 13.40% |
| Phenyl trimethicone (Mirasil PTM ®) | 47.20% |
| PVP/eicosene Copolymer (Antaron ® V220) | 4.00% |
| Diisostearyl malate (Salacos ® 222) | 17.90% |
| D&C Red 7 Calcium Lake (Rubine Lake C 19003 ®) | 5.18% |
| Titanium dioxide (titanium oxide W 877 ®) | 4.45% |
| D&C Red 33 Aluminum Lake (6533 D&C red AL lake ®) | 0.55% |
| D&C Red 7 Calcium Lake (Rubine Lake C 19025 ®) | 0.81% |
| FD&C Yellow No. 6 Aluminum Lake | 0.96% |
| (FD&C Yellow 6 Lake (C70.52.70)) | |
| Polymethylsilsesquioxane | 5.55% |
| (Tospearl 2000B+ ® or Tospearl 2000B*^{.} ®) | |

### Example 10

### Lipstick

| | |
|---|---|
| Ericerus Pela Wax (Refined Chinese Insect Wax ®) | 13.40% |
| Phenyl trimethicone (Mirasil PTM ®) | 47.20% |
| PVP/eicosene Copolymer (Antaron ® V220) | 4.00% |
| Diisostearyl malate (Salacos ® 222) | 17.90% |
| D&C Red 7 Calcium Lake (Rubine Lake C 19003 ®) | 5.18% |
| Titanium dioxide (titanium oxide W 877 ®) | 4.45% |
| D&C Red 33 Aluminum Lake (6533 D&C red AL lake ®) | 0.55% |
| D&C Red 7 Calcium Lake (Rubine Lake C 19025 ®) | 0.81% |
| FD&C Yellow No. 6 Aluminum Lake | 0.96% |
| (FD&C Yellow 6 Lake (C70.52.70)) | |
| Poly(glycol adipate)/bis(hydroxyethyoxypropyl) | 5.55% |
| dimethicone copolymer | |
| (Silslip Powder ®) | |

### Example 11

### Lipstick

| | |
|---|---|
| Ericerus Pela Wax (Refined Chinese Insect Wax ®) | 13.40% |
| Phenyl trimethicone (Mirasil PTM ®) | 47.20% |
| PVP/eicosene Copolymer (Antaron ® V220) | 4.00% |
| Diisostearyl malate (Salacos ® 222) | 17.90% |
| D&C Red 7 Calcium Lake (Rubine Lake C 19003 ®) | 5.18% |
| Titanium dioxide (titanium oxide W 877 ®) | 4.45% |
| D&C Red 33 Aluminum Lake (6533 D&C red AL lake ®) | 0.55% |
| D&C Red 7 Calcium Lake (Rubine Lake C 19025 ®) | 0.81% |
| FD&C Yellow No. 6 Aluminum Lake | 0.96% |
| (FD&C Yellow 6 Lake (C70.52.70)) | |
| Mica & Aluminum hydroxide | 5.55% |
| (Excel Mica JP2 ®) | |

### Example 12

### Eyeliner

| | |
|---|---|
| Ericerus Pela Wax (Refined Chinese Insect Wax ®) | 12.95% |
| Phenyl trimethicone (Mirasil PTM ®) | 45.70% |
| PVP/eicosene Copolymer (Antaron ® V220) | 3.85% |
| Diisostearyl malate (Salacos ® 222) | 18.00% |
| Brown iron oxide | 5.75% |
| Black iron oxide | 9.15% |
| Yellow iron oxide | 4.60% |

### Example 13

### Eyeshadow

| | |
|---|---|
| Ericerus Pela Wax (Refined Chinese Insect Wax ®) | 11.90% |
| Phenyl trimethicone (Mirasil PTM ®) | 41.90% |
| PVP/eicosene Copolymer (Antaron ® V220) | 3.50% |
| Diisostearyl malate (Salacos ® 222) | 18.00% |
| Chromium oxide | 3.50% |
| Ferric ferricyanide | 1.20% |
| Mica & titanium dioxide & ferric ferricyanide | 15.00% |
| Mica & silica & titanium dioxide | 4.00% |
| Mica & titanium dioxide | 1.00% |

### B. Measurement of the Penetration Index and the Breaking Index

For the cosmetic compositions 1 to 6 described previously, the penetration indices, on the one hand, and the breaking indices, on the other hand, were measured.

The penetration indices are measured using a P734 penetrometer from Normalab. Beforehand, mixtures corresponding to the preceding cosmetic compositions were poured into Petri dishes 1.2 cm deep and then placed at a temperature of 20° C. for 24 hours. The measurements are taken using an “18-0176 Unilever method” reference cone.

The breaking indices are measured using a TCM 201-M machine from Chatillon. Beforehand, mixtures corresponding to the preceding cosmetic compositions were poured into lipstick molds, so as to obtain casts with a diameter of about 1.3 cm. These casts are then placed at a temperature of 20° C. for 24 hours.

The results are collated in the following table;

| | Penetration index | Breaking index |
|---|---|---|
| Example | (10⁻¹ mm) | (g) |
| 1 | 36.4 | 703 |
| 2 | 37.7 | 603 |
| 3 | 61.6 | 255 |
| 4 | 40.6 | 455 |
| 5 | 26.1 | 807 |
| 6 | 21.6 | 1213 |

The lower the penetration index and/or the higher the breaking index, the harder the composition and the more difficult it is to break. It is found that composition 6 is the one that has the highest hardness, contrary to composition 3.

When the penetration index is less than 100×10⁻¹ mm, preferably less than 70×10⁻¹ mm and more preferentially less than 50×10⁻¹ mm, the product shows good stability.

When the breaking index is greater than 100 g, preferably greater than 200 g and more preferentially greater than 500 g, the product does not flow under its own weight and thus shows good stability.

### C. Compatibility Study for 17% Wax/83% Oil Systems and Penetration Index Measurements

Binary mixtures comprising Refined Chinese Insect Wax in combination with an oil as indicated in the tables below were prepared. These mixtures were poured into Petri dishes and placed at a temperature of 20° C. for 24 hours as described in Example B.

The penetration index measurements were taken as indicated in Example E.

| | | | |
|---|---|---|---|
| Brand of oil | Abil ® AV 20 | Mirasil ® PTM | Cyclomethi- |
| | from Degussa | from Rhodia | cone ® 5 |
| | | | from Dow |
| | | | Corning |
| INCI name of | Phenyl | Phenyl | Cyclopenta- |
| the oil | trimethicone | trimethicone | siloxane |
| Macroscopic | Homogeneous | Homogeneous | Homogeneous |
| appearance | gel | gel | gel |
| Penetration | 29.05 | 22.35 | 41.3 |
| index | | | |
| (10⁻¹ mm) | | | |

| | | |
|---|---|---|
| Brand of oil | Dimethicone ® 5 | DC ® 5562 carbinol fluid from |
| | from Dow | Dow Corning |
| | Corning | |
| INCI name of | Dimethicone | Bis(hydroxyethoxy)propyl |
| the oil | | dimethicone |
| Macroscopic | Homogeneous | Homogeneous gel |
| appearance | gel | |
| Penetration | 41.7 | 37.9 |
| index | | |
| (10⁻¹ mm) | | |

It is thus found that the composition with cyclopentasiloxane or dimethicone is softer than that comprising phenyl trimethicone.

Nevertheless, all these mixtures show good stability and do not flow under their own weight. These results confirm that a high percentage of silicone oil can be included in a solid cosmetic composition by virtue of the presence of Chinese insect wax.

### D. Sensory Analysis

A sensory analysis on the formulation of Example 1 was performed on a panel of 23 individuals. The lipsticks were tested under standardized conditions (controlled temperature, hygrometry and light).

Each individual applies the formulation to her entire lips a first time, then makes two further applications. Four minutes later, each individual applies a paper handkerchief to her lips, and 15 minutes later each individual moistens/wets her lips three times.

During its application, the lipstick has a very slippery and very fondant texture. The deposit during the first pass onto the lips is of low thickness.

The lipstick gives a uniform, opaque, matt makeup result of moderate color intensity with a very light pearlescent effect. It allows a sharp contour to be drawn.

After its application, the lipstick leaves the lips non-tacky and very soft. In addition, in terms of immediate staying power, it does not run and does not come off onto a paper handkerchief.

### E. Comparative Experiment

A comparative experiment was conducted to evaluate the ability of Chinese insect wax to structure high amounts of silicon oil, compared to other waxes typically used in cosmetic products.

To this end, the lipstick of Example 4 (using MIRASIL® PTM instead of ABIL® AV20 as phenyl trimethicone) was compared with lipsticks having the same constituents in the same amounts, except that carnauba wax and beeswax were substituted for Chinese insect wax, to yield comparative compositions 4A and 4B, respectively.

The macroscopic aspect of the products made from Compositions 4, 4A and 4B was then assessed. Composition 4 resulted in an homogeneous and hard product, whereas the product obtained from Composition 4A was hard but heterogeneous, with the presence of a waxy white coating, and the product obtained from Composition 4B was soft and heterogeneous, wherein some of the nacres had sedimented. This latter product had a Breaking Index that was 17 times lower than that measured for the product obtained from Composition A.

This example demonstrates that only Chinese insect wax is able to properly structure high amounts of silicon oils.

1-14. (canceled)

15. A cosmetic composition in solid form comprising from 25% to 90%, relative to the weight of the total composition, of at least one silicone oil and an extract of Ericerus pela wax.

16. The composition as claimed in claim 15, wherein it comprises from 30% to 80%, relative to the weight of the total composition, of at least one silicone oil.

17. The composition as claimed in claim 15, wherein it comprises from 40% to 75%, relative to the weight of the total composition, of at least one silicone oil.

18. The composition as claimed in claim 15, wherein the extract of Ericerus pela wax represents from 5% to 35% by weight relative to the weight of the total composition.

19. The composition as claimed in claim 15, wherein the extract of Ericerus pela wax represents from 10% to 20% by weight relative to the weight of the total composition.

20. The composition as claimed in claim 15, wherein the silicone oil is a glossy oil chosen from phenyl silicones, an alkyl siloxysilicate, and mixtures thereof.

21. The composition as claimed in claim 15, wherein the silicone oil is chosen from phenyl trimethicone, phenylpropyldimethyl siloxysilicate and bis(hydroxyethoxy)propyl dimethicone, and mixtures thereof.

22. The composition as claimed in claim 15, wherein it also comprises at least one ester chosen from isononyl isononanoate, diisostearyl malate, octyldecyl neopentanoate and 2-ethylhexyl palmitate, and mixtures thereof.

23. The composition as claimed in claim 15, wherein it is in cast form.

24. The composition as claimed in claim 15, wherein it is in the form of a stick.

25. The composition as claimed in claim 15, wherein it is in anhydrous form.

26. The composition as claimed in claim 15, wherein it is in the form of an oil-in-water emulsion, a water-in-oil emulsion, a multiple emulsion or a hydrodispersion.

27. The composition as claimed in claim 15, wherein it also comprises at least one hydrocarbon-based oil and/or at least one pigment.

28. A cosmetic process for caring for and/or making up keratinous matter, wherein it comprises the application to the keratinous matter of a cosmetic composition in accordance with claim 15.

29. A process according to claim 28, wherein the composition is applied in the form of a pencil, mascara, eyeliner, foundation, lip gloss, lipstick, lipcare stick, body makeup product, eyeshadow, makeup rouge, concealer product, facial care product or body care product.
